(19) United States
(12) Patent Application Publication
(10) Pub. No.: US 2002/0100481 A1
(43) Pub. Date: Aug. 1, 2002

(54) Eye shield

(75) Inventor: Qamar Abbasi, Dublin (IE)

Correspondence Address: SUGHRUE, MION, ZINN, MACPEAK & SEAS, 2100 Pennsylvania Avenue, N.W., Washington, DC, 20037-3202, US

(73) Assignee: QAMAR ABBASI

(21) Appl. No.: 10/103,824

(22) Filed: Mar. 25, 2002

Related U.S. Application Data
(63) Continuation of application No. 09/743,418, filed on Mar. 9, 2001.
Application No. 09/743,418 is a 371 of international application No. PCT/IE99/00063, filed on Jul. 12, 1999.

(30) Foreign Application Priority Data
Jul. 10, 1998 (IE) S980556

(51) Int. Cl. A61F 9/00

(52) U.S. Cl. 128/858

(57) ABSTRACT

An eye shield (1) for retaining an eye (2) of an anaesthetized subject closed during a surgical procedure for protecting the eye from injury comprises a patch (3) of substantially oval shape having a rear major surface (7). An upper adhesive portion (14) and a lower adhesive portion (15) of the rear major surface (7) are coated with a self-adhesive coatings (11,12) for securing the patch (3) to an upper eyelid (9) and a lower eyelid (10), respectively. An adhesive tab (16) extends downwardly from the lower adhesive portion (15) for securing the patch (3) to a portion (17) of the face of the subject below the eye (2). A non-adhesive portion (18) extends along a major axis (5) between borders (37,38) of the upper and lower adhesive portions (14,15), respectively, for preventing adhesion of the patch (3) to eyelashes of the upper and lower eyelids (9,10). A substantially transparent window (24) is provided in the patch (3) for facilitating viewing of the open/closed status of the eye (2) during the surgical procedure.

The present invention relates to an eye shield for protecting an eye of a subject, and in particular, to an eye shield for protecting an eye of a subject during a surgical procedure when the subject is anaesthesised, unconscious or sedated. The invention also relates to a method for protecting an eye of a subject during a surgical procedure when the subject is anaesthesised, unconscious or sedated.

When a subject is anaesthesised, unconscious or sedated during a surgical procedure, or indeed, at any time while a subject is being attended to when anaesthesised, unconscious or sedated, the eyes of the subject may open. In such a state the subject is vulnerable to injury and/or irritation to the eyes. The most common type of injury suffered by anaesthesised subjects during non-ocular surgery is corneal abrasion. Other types of eye injury and irritation which may be suffered by an anaesthesised patient undergoing non-ocular surgery include conjunctivitis, red eyes, blurred vision, chemical injury, eyelid haematoma, and in extreme cases permanent loss of vision. Such eye injuries and irritations may be caused by direct trauma, by chemicals inadvertently coming into contact with the eye, and by lagopthalmus which can lead to drying of the eyes and corneal abrasion. Corneal abrasion typically is caused by a surgical gown of a surgeon, or other items which depend from a surgeon inadvertently being wiped across the eyes of the subject. Indeed, it is not unknown for eye abrasion to be caused by watchstraps on the arm of a surgeon or an anaesthetist. Chemical injuries may result from chemicals used during the surgical procedure inadvertently coming into contact with the eyes of the subject. It is well known that anaesthetic gases and skin preparations used in the preparation of a subject prior to an operation can cause irritation of the eyes. Indeed, the surgical positioning of the head and neck can reduce venous retum, thus, leading to corneal edema. Additionally tear production is significantly reduced during general anaesthesis thus leading to dryness of the eye. Needless to say, the longer the surgical procedure lasts, the risk of eye injury and irritation increases. Additionally, there is a significant reduction in the bell phenomenon during general anaesthesia. The bell phenomenon is the phenomenon of natural random motion of the eye during sleep. This eye activity has been found to cease or to dramatically decrease during sedation, anaesthesia and unconsciousness, thereby, fixing the eyes in a normal straight ahead position in which the eyes are most vulnerable to injury. Studies of such injuries include Terry H. R., Keams T. P, Love J. G. and Orwell G., “Untoward and neurological events of anaesthesia”, Surg Cli N Am 45:937-937,1965.

Although eye injuries which occur during anaesthesia for non-ocular surgical procedure are relatively uncommon, nonetheless, when such injuries do occur, they lead to significant discomfort, pain and in extreme cases loss of vision. See for example Cucchiara R. F., Black: Corneal Abrasion during anaesthesia and surgery, ANAESTHESIOLOGY1988:69:978-9). Furthermore, eye injuries during non-ocular surgical procedures, in general, tend to lead to relatively high monetary settlements, compared with claims for non-ocular injuries, see for example, “A study of the American Society of Anaesthesiologists”, in particular, an article by Gild W. M., Posner K. L., Caplan R. A., Cheney F. W.: “Eye injuries associated with anaesthesia, a closed claim analysis”. Anaesthesiology 1992; 76:204-8. In this study corneal abrasion has been shown to be the most common eye injury. One literature reference, Steven R, Ronald A T et al, “Eye injuries after non-ocular surgery-A study of 660,965 Anaesthetics from 1988-1992”, ANAESTHESIOLOGY, 1996, 85:1020-7 concludes that the most common cause of corneal abrasion was accidental loosening or removal of tape coverings of eyes leading to exposure keratopathy. A further study, E. White, M M Crosse,: “The aetiology of preoperative corneal abrasions” (Review Article) Anaesthesia, 1998, 53, pages 157-161) concludes that the routine use of aqueous solution, viscous gels and ointments during preoperative period does not offer sufficient additional protection against the development of corneal abrasion. This study also found that ointments in particular contributed to significant morbidity. The routine use of the solutions, gels and ointments was not recommended by this study.

Other related publications included:

Batra Y K, Bali Im, “Corneal abrasion during general anaesthesia”, ANAESTHESIA AND ANALGESIA, 1977; 56:363-5;

Saude T, “Ocular Anatomy and Physiology” Oxford; Blackwell Scientific Publication, 1990;

Cucchiara R F, Lack S, “Corneal abrasions during anaesthesia and surgery” ANAESTHESIOLOGY 1988; 69:978-979;

Boggild-Madsen N B, Bungarrd-Neilsen P, Hammer U, Jackbson B, “Comparison of Methylcellulose and paraffin ointments during general anaesthesia”, CANADIAN ANAESTHETIST'S SOCIETY JOURNAL 1981; 28:575-8;

Green S, Woodwin H, Moss J, comps, “Risk Management in Anaesthesia” London, THE MEDICAL DEFENCE UNION LIMITED, 1997.

Various methods are used for retaining the eyes of an anaesthetised subject closed during surgical procedures. For example, it is known to apply a strip of adhesive tape across each eye from the eyebrow, downwardly, across the upper and lower eyelids and to the face of the subject below the eye. Each strip of adhesive tape is thus, secured to the eyelids from the eyebrows to a portion of the face below the respective eyes. Various types of adhesive tapes are available, for example, a paper tape sold under the Trade Mark MICROPORE, and a plastics film tape sold under the Trade Mark TRANSPORE both of 3M. Both of these tapes are porous, and thus permeable to chemicals and other solutions used during surgical procedures and in the preparation of a subject for a surgical procedure. Furthermore, the adhesive of these tapes is relatively weak, and thus, the tapes are vulnerable to being displaced during a surgical procedure. Another type of tape is a linen tape sold under the Trade Mark DERMICEL by Johnson & Johnson. This tape is also porous, and thus permeable to chemicals used in surgical procedures and in the preparation of a subject for a surgical procedure. However, a particularly serious problem with this tape is that the adhesive is relatively strong, and thus removal of the tape after the surgical procedures causes considerable discomfort to the subject, and in many cases loss of hairs of the eyebrows and of the eyelashes, which may be plucked during removal of the tape. There are other paper, plastics film, linen and gauze tapes available, however, all suffer from one or more of the above disadvantages. In the case of tapes with relatively strong adhesive, it is known to use gels and ointments, such as those sold under the Trade Marks LACRILUBE and DURATEARS to reduce the adhesive properties of the adhesive tapes. Methycellulose drops and hydrophillic contact lenses also have been used in conjunction with tapes to minimise the affects of those tapes with relatively strong adhesive. However, the use of such gels, ointments and drops unfortunately, by reducing the adhesive properties of the adhesive can lead to the tapes being inadvertently displaced during the procedure. This, can lead to opening of the eyes during the surgical procedure with the consequent risk of eye injury and irritation. It is also not unknown for the adhesive on many adhesive tapes to cause skin irritation. Needless to say, the amount of irritation can vary from the type of tape used and the amount of adhesive on the tape.

U.S. Pat. No. 4,649,908 of Ghaly discloses an eye shield device for protecting the eyes of an anaesthesised subject during a non-ocular surgical procedure. The eye shield comprises at least one soft, pliable air-filled cushion member of thin plastics material, sized and shaped to fit within the cavity formed by the orbital bone structure around the eye. The cushion member is to be placed upon the eye when closed, and is secured in place by an eye shield support which is to be removably affixed to the head of the individual. It is believed that this eye shield device has never been used, and it is believed it would be unsatisfactory, in that it would not always retain the eye of the subject closed, it is large, bulky and cumbersome, and would be prone to being inadvertently displaced during a surgical procedure.

There is therefore a need for a relatively simple, yet effective eye shield for retaining an eye of a subject closed and to shield and protect the eye from injury, particularly, when the subject is anaesthesised, sedated or unconscious or other procedure.

According to the invention there is provided an eye shield for protecting an eye of a subject, the eye shield comprises a patch having a rear major surface for in use abutting at least an upper eyelid of an eye of the subject, and a front major surface, an adhesive means on the rear major surface for securing the patch to the upper eyelid and to a lower eyelid or a portion of the face of the subject below the eye for retaining the eyelids closed, and a viewing means for facilitating viewing of the open/closed status of the eyelids.

Preferably, a portion of the rear major surface of the patch defines an intermediate area for in use coinciding with eyelashes of the subject, the intermediate area being such as to facilitate removal of the eye shield without plucking of the eyelashes, and with minimum discomfort to the subject. Advantageously, the intermediate area is provided by a non-adhesive surface for preventing adhesion of the patch to eyelashes of the respective upper and lower eyelids.

Advantageously, the adhesive means is provided on an upper portion of the rear major surface which defines an upper adhesive portion for securing the patch to the upper eyelid intermediate the eyebrow and eyelash, and the adhesive means is provided on a lower portion of the rear major surface which defines a lower adhesive portion for securing the patch to the lower eyelid or to the face of the subject below the eye, the intermediate area being located between the upper and lower adhesive portions.

In one embodiment of the invention the adhesive means is located on the lower adhesive portion of the rear major surface for securing the patch below the eyelash of the lower eyelid.

Ideally, the intermediate area extends from one side edge to another opposite side edge of the patch between the upper and lower adhesive portions thereof.

In another embodiment of the invention the lower adhesive portion of the patch comprises an adhesive tab extending downwardly therefrom for securing to the face of the subject below the eye.

In an alternative embodiment of the invention the adhesive tab extends obliquely downwardly from the patch. In a further alternative embodiment of the invention a pair of adhesive tabs diverging outwardly from each other extend downwardly from the patch.

Preferably, the upper adhesive portion defines an area which is substantially similar to the shape of an upper eyelid when dosed.

Advantageously, a non-adhesive tab extends from the patch, and preferably, extends upwardly from the patch for facilitating removal of the patch from the eye, and preferably, in a direction from the upper eyelid downwardly towards the lower eyelid for minimising discomfort to the subject during removal thereof.

Preferably, the viewing means is located between the upper and lower adhesive portions. Advantageously, the viewing means comprises a window of a substantially transparent material.

In one embodiment of the invention the window defines a part of the rear major surface of the patch, the rear surface of the window defining at least a part of the intermediate area of the rear major surface.

In another embodiment of the invention the window is framed by the patch material.

In a still further embodiment of the invention the viewing means defines an area which is substantially similar to the shape of a lower eyelid.

Preferably, the patch is of an opaque material.

Ideally, the patch defines an outer periphery which is of substantially similar shape to the oval outline shape of an eye cavity. Preferably, the oval shaped patch defines a major axis, which in use extends substantially parallel to a line defined by the abutting edges of the upper and lower eyelids of the eye when closed.

In one embodiment of the invention the patch defines an upper straight edge for preventing contact of the patch with the eyebrow of the eye. Advantageously, the upper straight edge of the patch extends parallel to the major axis of the patch.

In one embodiment of the invention the viewing means defines an upper straight edge which extends parallel to the major axis of the patch. Advantageously, the upper straight edge of the viewing means lies above the major axis of the patch, and ideally, the viewing means is of semi-oval shape, extending downwardly from the upper edge thereof.

In one embodiment of the invention the patch is of substantially similar size to the size of the eye cavity.

In another embodiment of the invention a protective release sheet is provided for covering and protecting the adhesive means. Preferably, the protective release sheet is provided in two parts which meet adjacent the major axis of the patch, and adjacent edges of the release sheets extend parallel to the major axis of the patch. Advantageously, the respective release sheets overlap adjacent the major axis of the patch.

In one embodiment of the invention the adhesive means is a self adhesive provided on the respective upper and lower adhesive portions.

In another embodiment of the invention the adhesive means is a non-irritant adhesive.

In a further embodiment of the invention the adhesive means is a medical grade adhesive.

Ideally, the patch is of a flexible material. Preferably, the patch is of a relatively thin film material.

Preferably, the patch and the window are of a material which is substantially impermeable to chemicals and other solutions used in surgical procedures and in the preparation of a subject for a surgical procedure.

Further, the invention provides a pair of eye shields according to the invention.

Additionally, the invention provides an eye shield for protecting an eye of a subject, the eye shield comprises a patch defining an outer periphery which is of substantially similar shape to the oval outline of the shape of an eye cavity, and having a rear major surface for in use abutting at least an upper lid of an eye of the subject, and a front major surface, an adhesive means on the rear major surface for securing the patch to the upper eyelid and to a lower eyelid or the portion of the face of the subject below the eye for retaining the eyelids close.

Further the invention provides a method for protecting an eye of a subject during a surgical procedure comprising the steps of securing a patch to an upper lid of the eye of the subject, urging the upper lid downwardly for dosing the eye and securing the patch to the lower lid of the eye, or to a portion of the face of the subject below the eye for retaining the upper and lower lids of the eye closed.

Preferably, the method further comprises the step of inspecting the eyelids through a viewing means in the patch for determining the open/closed status of the eyelids.

Advantageously, the method further comprises preventing securing of the patch to eyelashes of the respective upper and lower eyelids when securing the patch to the upper eyelid and to the lower eyelid or the portion of the face below the eye.

Ideally, the method comprises securing the patch to the upper eyelid intermediate the eyelash of the upper eyelid and the eyebrow of the eye of the subject.

In one embodiment of the invention the method further comprises the step of removing the patch from the eye by peeling the patch from the upper eyelid in a direction from the upper eyelid downwardly towards the lower eyelid.

Additionally, the method comprises the step of securing a pair of patches to the respective eyes of the subject, and ideally, the patches are provided by respective eye shields according to the invention.

The advantages of the invention are many. The eye shield according to the invention provides a simple yet effective shield for maintaining an eye of anaesthetised subject closed during a non-ocular surgical procedure, thereby protecting the eye from injury and irritation. By providing the intermediate area as a non-adhesive portion on the patch intermediate the upper and lower adhesive portions adhesion of the patch to the eyelashes is avoided, and thus there is no danger of injury or plucking of the eyelashes during removal of the patch. Additionally, by virtue of the fact that the patch is adapted for securing to the upper eyelid between the eyelashes of the upper lid and the eyebrow, there is similarly no danger of the patch adhering to the eyebrows, and thus no danger of plucking of the eyebrows during removal of the patch. The eye shield may readily easily be provided as a disposable shield, and thus, can readily easily be disposed of. By providing the patch in a substantially oval-shape similar in shape and size to the oval outline of the eye cavity, the patch can readily easily be aligned with the eye during fixing of the patch to the upper and lower eyelids and to the portion of the face below the eye. An additional advantage of the eye shield according to the invention is that the eye shield can be maintained relatively easily free of debris since the patch can be affixed to the eye without the need for touching the adhesive portion as is common when applying tape. The provision of the viewing means in the form of a window or otherwise allows the open/closed status of the eye to be viewed without the need to remove the patch from the eye.

Additionally, by minimising the adhesive portions, in other words, by providing only upper and lower adhesive portion the exposure of the skin to the adhesive is minimised, thus, minimising the risk of allergic reaction to the adhesive. The risk of allergic reaction can be further reduced by the use of an hypo-allergenic medical grade adhesive. Additionally, by providing upper and lower adhesive portions, the upper adhesive portion can be shaped to correspond substantially to the shape of an upper eyelid, while the lower adhesive portion can be shaped to correspond to a lower eyelid when the eyelids are dosed.

The invention will be more clearly understood from the following description of some preferred embodiments thereof which are given by way of example only with reference to the accompanying drawings, in which:

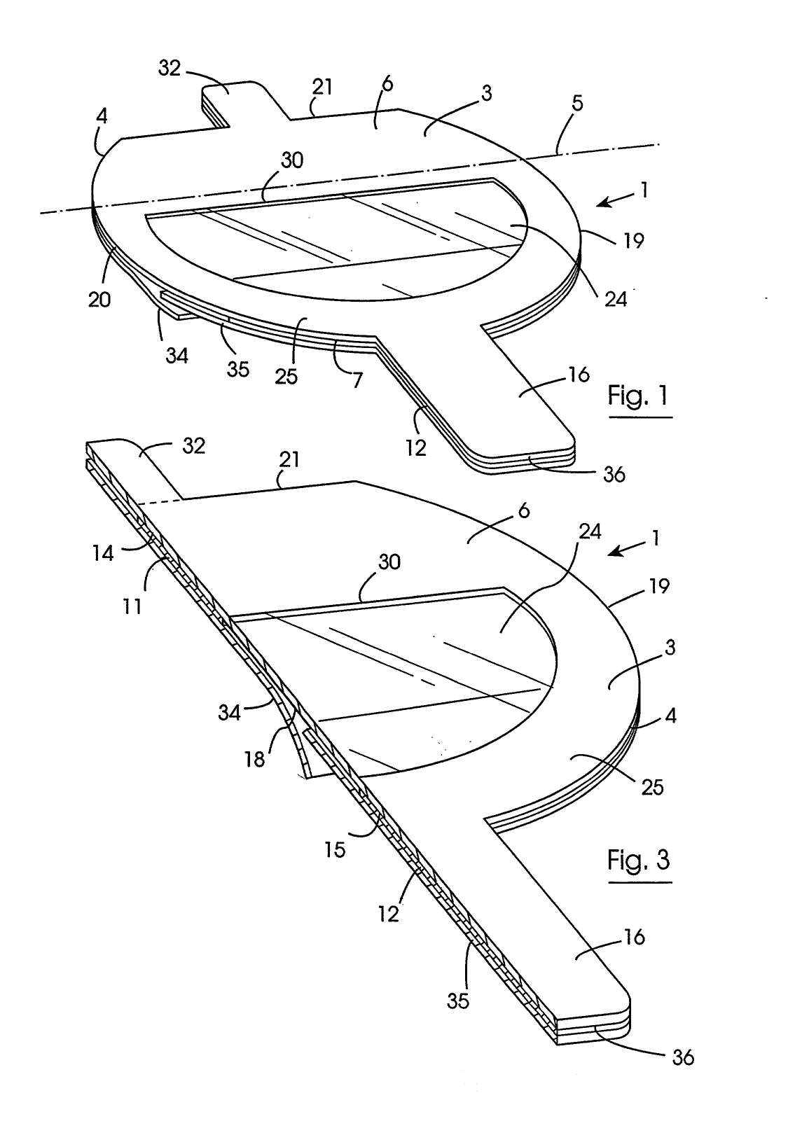

FIG. 1 is a perspective view of an eye shield according to the invention,

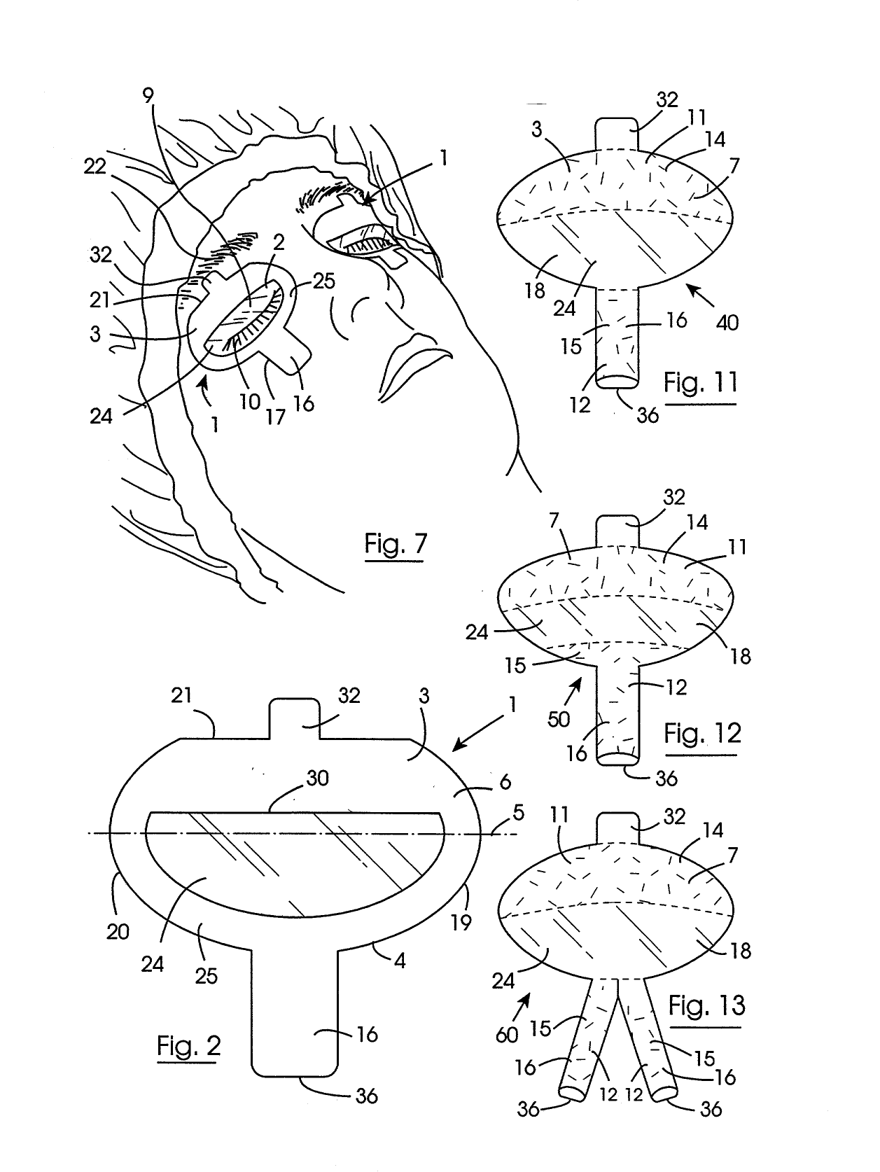

FIG. 2 is a top plan view of a the eye shield of FIG. 1,

FIG. 3 is a sectional perspective view of the eye shield of FIG. 1,

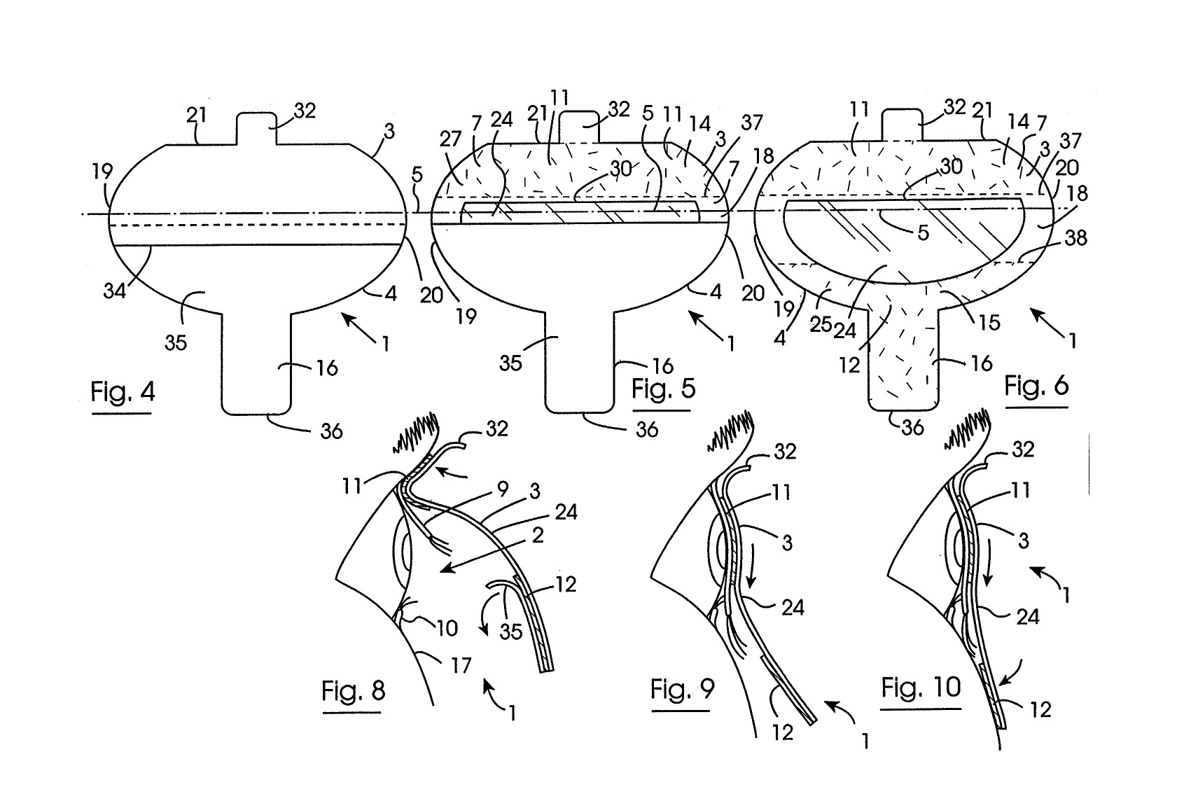

FIG. 4 is an underneath plan view of the eye shield of FIG. 1,

FIG. 5 is an underneath plan view of the eye shield of FIG. 1 with a portion of the eye shield removed,

FIG. 6 is an underneath plan view of the eye shield of FIG. 1 with another portion of the eye shield removed,

FIG. 7 is a perspective view of a pair of the eye shields of FIG. 1 in use,

FIG. 8 is a side elevational view of the eye shield of FIG. 1 in use,

FIG. 9 is a side elevational view of the eye shield of FIG. 1 in a different position to FIG. 8 in use,

FIG. 10 is a side elevational view of the eye shield of FIG. 1 in a still further different position in use,

FIG. 11 is an underneath plan view of an eye shield according to another embodiment of the invention,

FIG. 12 is an underneath plan view of an eye shield according to a further embodiment of the invention, and

FIG. 13 is an underneath plan view of an eye shield according to a still further embodiment of the invention.

Referring to the drawings and initially to FIGS. 1 to 10 thereof there is illustrated a disposable eye shield according to the invention indicated generally by the reference numeral 1 for protecting an eye 2 of a subject, and for retaining the eye closed during a non-ocular surgical procedure while the subject is under anaesthesia. The eye shield 1 comprises a patch 3 of relatively thin flexible ethylene vinyl acetate opaque film. In this embodiment of the invention, the patch 3 is of substantially oval shape having an outer periphery 4 which substantially defines the oval outline of the eye cavity in shape and size, and defines a major axis 5. The patch 3 has a front major surface 6 and a rear major surface 7 for abutting upper and lower eyelids 9 and 10, respectively, of the eye 2. An adhesive means provided by self-adhesive coatings 11 and 12 are provided on an upper adhesive portion 14 and a lower adhesive portion 15, respectively, of the rear major surface 7 for securing the patch 3 to the upper eyelid 9 and the lower eyelid 10, or a portion 17 of the face of the subject below the eye 2, respectively, and for retaining the upper and lower lids 9 and 10, respectively, of the eye 2 closed. An adhesive tab 16 extends downwardly from the lower adhesive portion 15 for securing the patch 3 to the portion 17 of the face of the subject below the eye 2. An intermediate area, in this embodiment of the invention provided by a non-adhesive portion 18 of the rear major surface 10 is located between the upper and lower portions 14 and 15 and extends along the major axis 5 between side edges 19 and 20 of the patch 3 for preventing adhesion of the patch 3 to eyelashes of the upper and lower eyelids 9 and 10. The upper adhesive portion 14 of the patch 3 terminates in an upper substantially straight edge portion 21 so that the patch 3 clears the eyebrow 22 of the eye 2 for avoiding adhesion of the upper adhesive portion 14 to the eyebrow 22.

A viewing means comprising a window 24 of substantially transparent flexible film is provided within the patch 3 for facilitating viewing of the open/closed status of the eye during the surgical procedure. In this embodiment of the invention the window 24 is also of ethylene vinyl acetate film and forms part of the front and rear major surfaces 6 and 7, and forms part of the non-adhesive portion 18. The window 24 is framed by the patch 3, and a strip 25 of the patch 3 part of which forms the lower adhesive portion 15 of the patch 3 extends around the lower portion of the window 24. The window 24 is semi-oval shaped having an upper edge 30 which extends parallel to the major axis 5 of the patch 3 and lies above the major axis 5, and extends downwardly from the upper edge 30. The term substantially transparent as used herein means that the window 24 is sufficiently transparent to allow viewing of the open/closed status of the eye without the need to remove the eye shield.

A non-adhesive tab 32 extends upwardly from the upper adhesive portion 14 for facilitating removal of the patch 3 from the upper eyelid 9 in a direction downwardly towards the lower eyelid 10. A pair of release sheets 34 and 35 protect the adhesive coatings 11 and 12, respectively, on the upper adhesive portion 14 and the lower adhesive portion 15 and the adhesive tab 16 until the patch is ready for use. The release sheets 34 and 35 overlap adjacent the major axis 5 and extend from a position adjacent the major axis 5 to the straight edge 21 of the upper adhesive portion 14 and to a tip 36 of the adhesive tab 16, respectively.

In this embodiment of the invention the adhesive coating 11 is provided in the upper adhesive portion 14 within the area bound by the upper straight edge 21, the side edges 19 and 20 and the broken line 37. The adhesive coating 12 is provided on the lower adhesive portion 15 on the adhesive tab 16 and on the strip 25 below the broken line 38.

In this embodiment of the invention the portion of the patch 3 which forms the upper portion 14, the strip 25 and the adhesive tab 16 as discussed above are of opaque ethylene vinyl acetate film. The film forming the window 24 and the film of the patch 3 are both relatively thin medical grade film. The upper and lower adhesive coatings 11 and 12 are provided by respective continuous coatings of a non-irritant medical grade adhesive. Although, if desired, the adhesive coatings 11 and 12 may be non-continuous and may be provided by a series of dots or strips of adhesive. The area of the upper adhesive portion 14 coated with the adhesive coating 11 is of shape substantially similar to the shape of the upper eyelid, and the shape of the area of the lower adhesive portion 15 coated with the adhesive coating 12 is of shape substantially similar to the shape of the lower eyelid for facilitating affixing the eye shield 1 to the subject. The non-adhesive tab 32 is also of ethylene vinyl acetate and is formed from the ethylene vinyl acetate film of the patch 3. The release sheets 34 and 35 are of paper material.

The dimensions of the eye patch for an adult are approximately 60 mm along the major axis 5, and 35 mm from the upper straight edge 21 to the lowest edge of the oval part of the patch 3. The adhesive tab 16 is approximately 20 mm by 15 mm. The corresponding dimension of the patch which would be suitable for a child are approximately 45 mm along the major axis 5, 30 mm from the upper straight edge 21 to the lower edge of the oval shaped patch 3, and the adhesive tab 16 approximately 20 mm by 15 mm. Alternatively, the patch may be provided in one size which would be suitable for both adults and children.

Typically, the eye shield 1 will be sold in pairs and may be individually packed or packed in pairs in sterile packs or may be clean room packaged.

In use, the eye shield 1 is affixed to the eye 2 as follows. Initially, the release sheet 34 which overlaps the release sheet 35 is removed and the patch 3 is offered up to the eye. The upper adhesive portion 14 is applied to the upper eyelid 9 and is located between the eyebrow 22 and the eyelashes of the upper eyelid 9 and secured to the upper eyelid 9. The release sheet 35 is then removed and the patch 3 is urged downwardly for ensuring that the upper eyelid 9 is closed and abutting the lower eyelid 10. In this position the lower adhesive portion 15 is secured to the lower eyelid and the portion 17 of the face of the subject below the eye. The portion of the strip 25 which carries adhesive is secured to the lower eyelid 10 and the adhesive strip 16 is secured to the portion 17 of the face below the eye 2. Care is taken during affixing of the patch 3 to the upper and lower eyelids 9 and 10 to ensure that only the non-adhesive portion 18 of the rear major surface 7 abuts the eyelashes of the upper and lower eyelids 9 and 10.

To remove the patch 3 from the eye 2 the non-adhesive tab 32 is gripped and the upper adhesive portion 14 is urged from the upper eyelid 9. The patch 3 is progressively urged from the eye 2 downwardly from the upper eyelid 9, from the lower eyelid 10 and from the portion 17 of the face below the eye 2.

Referring now to FIG. 11 there is illustrated an eye shield 40 according to another embodiment of the invention for protecting an eye of an anaesthetised subject and retaining the eye closed during a surgical procedure. The shield 40 is substantially similar to the shield 1 and similar components are identified by the same reference numerals. The main difference between the shield 40 and the shield 1 is that instead of the window 24 being framed by the patch 3, the upper adhesive portion 14 and the lower adhesive portion 15 which is formed by the adhesive tab 16 are separated from each other and joined by the window 24. The window 24 in this embodiment of the invention provides the non-adhesive portion 18. Otherwise, the eye shield 40 is substantially similar to the eye shield 1.

Referring now to FIG. 12 there is illustrated an eye shield 50 according to a further embodiment of the invention. The eye shield 50 is substantially similar to the eye shield 1 and similar components are identified by the same reference numerals. The main difference between the eye shield 50 and the eye shield 1 is that like the eye shield 40 the upper and lower adhesive portions 14 and 15 are separated by the window 24 and are joined by the window 24. The window 24 in this embodiment of the invention also forms the non-adhesive portion 18. However, in this embodiment of the invention the adhesive tab 16 which extends downwardly from the window 24 extends obliquely relative to the major axis 5 for facilitating attachment of the adhesive tab 16 to the portion 17 of the face of the subject below the eye 2 and adjacent the nose. Otherwise, the eye shield 50 is similar to the eye shield 1.

Referring now to FIG. 13 there is illustrated an eye shield 60 according to a still further embodiment of the invention. The eye shield 60 is substantially similar to the eye shield 1, and similar components are identified by the same reference numerals. In fact, the eye shield 60 is identical to the eye shield 50 with the exception that instead of one obliquely extending adhesive tab 16, a pair of obliquely extending adhesive tabs 16 are provided which diverge downwardly from each other. In this embodiment of the invention it is envisaged that each adhesive tab 16 may be provided with a separate release sheet which may be removed independently of each other, and in which case it is envisaged that initially only one of the release sheets would be removed for securing one of the adhesive tabs 16 to the face of the subject. The release sheet on the other adhesive tab 16 would only be removed in the event of loss of adhesiveness by the adhesive tab 16 which is in use.

It is envisaged that in certain cases, the eye shields according to the invention may be provided without the window, and without any other viewing means, in which case, the eye shields would be formed from a single film of opaque material. Alternatively, it is envisaged that the eye shield may be provided by a single sheet of substantially transparent material.

While it is preferable, that a non-adhesive portion coinciding with the eyelashes of the subject be provided for preventing the patch adhering to the eyelashes, it is envisaged in certain cases, that such a non-adhesive portion may be omitted, provided that the adhesive is of a strength which may be removed without plucking the eyelashes and without discomfort to the subject.

Additionally, while the intermediate area has been described as being a non-adhesive portion of the rear major surface for preventing adhesion of the patch to the eyelashes, it is envisaged in certain cases that the intermediate area may be provided with an adhesive of weaker strength than the adhesive provided on the upper and lower adhesive portions. Indeed, the intermediate area may be coated with an adhesive similar to the adhesive on the upper and lower adhesive portions, provided that the adhesive would have adequate properties for retaining the patch attached to the upper eyelid and to the lower eyelid or the portion of the face below the eye, while at the same time the adhesive properties of the adhesive would be such as to present plucking of the eyelashes and discomfort to the subject during removal of the eye shield.

It is envisaged that the eye shield may be of any other suitable material, which preferably, should be a medical grade material, for example, polyethylene or the like. Needless to say, it will be appreciated that the adhesive tab may be omitted. It will also be appreciated that the release tab may be located in any other suitable location.

1. An eye shield (1) for protecting an eye (2) of a subject, characterised in that the eye shield (1) comprises a patch (3) having a rear major surface (7) for in use abutting at least an upper eyelid of an eye of the subject, and a front major surface (6), an adhesive means (11,12) on the rear major surface (7) for securing the patch (3) to the upper eyelid and to a lower eyelid or a portion of the face of the subject below the eye for retaining the eyelids dosed, and a viewing means (24) for facilitating viewing of the open/closed status of the eyelids.

2. An eye shield as claimed in claim 1 characterised in that the apportion of the rear major surface of the patch (3) defines an intermediate area (18) for in use coinciding with eyelashes of the subject, the intermediate area (18) being such as to facilitate removal of the eye shield without plucking of the eyelashes, and with minimum discomfort to the subject.

3. An eye shield as claimed in claim 2 characterised in that the intermediate area (18) is provided by a non-adhesive surface (18) for preventing adhesion of the patch (3) to eyelashes of the respective upper and lower eyelids.

4. An eye shield as claimed in claim 2 or 3 characterised in that the adhesive means (11) is provided on an upper portion of the rear major surface (7) which defines an upper adhesive portion (14) for securing the patch (3) to the upper eyelid intermediate the eyebrow and eyelash, and the adhesive means (12) is provided on a lower portion of the rear major surface (7) which defines a lower adhesive portion (15) for securing the patch (3) to the lower eyelid or to the face of the subject below the eye, the intermediate area (18) being located between the upper and lower adhesive portions (14,15).

5. An eye shield as claimed in claim 4 characterised in that the adhesive means (12) is located on the lower adhesive portion (15,16) of the rear major surface (7) for securing the patch (3) below the eyelash of the lower eyelid.

6. An eye shield as claimed in any of claims 4 or 5 characterised in that the intermediate area (18) extends from one side edge (19) to another opposite side edge (20) of the patch (3) between the upper and lower adhesive portions (14,15) thereof.

7. An eye shield as claimed in any of claims 4 to 6 characterised in that the lower adhesive portion (15) of the patch (3) comprises an adhesive tab (16) extending downwardly therefrom for securing to the face of the subject below the eye.

8. An eye shield as claimed in claim 7 characterised in that the adhesive tab (16) extends obliquely downwardly from the patch.

9. An eye shield as claimed in claim 7 or 8 characterised in that a pair of adhesive tabs (16) diverging outwardly from each other extend downwardly from the patch.

10. An eye shield as claimed in any of claims 4 to 9 characterised in that the upper adhesive portion (14) defines an area which is substantially similar to the shape of an upper eyelid when closed.

11. An eye shield as claimed in any preceding claim characterised in that a non-adhesive tab (32) extends from the patch (3), and preferably, upwardly from the patch (3) for facilitating removal of the patch (3) from the eye, preferably, in a direction from the upper eyelid downwardly towards the lower eyelid for minimising discomfort to the subject during removal thereof.

12. An eye shield as claimed in any preceding claim characterised in that the viewing means (24) is located between the upper and lower adhesive portions (14,15).

13. An eye shield as claimed in any preceding claim characterised in that the viewing means (24) comprises a window (24) of a substantially transparent material.

14. An eye shield as claimed in claim 13 characterised in that the window (24) defines a part of the rear major surface (7) of the patch, the rear surface of the window defining at least a part of the intermediate area (18) of the rear major surface (7).

15. An eye shield as claimed in claim 13 or 14 characterised in that the window (24) is framed by the patch material (3,25).

16. An eye shield as claimed in any preceding claim characterised in that the viewing means (24) defines an area which is substantially similar to the shape of a lower eyelid.

17. An eye shield as claimed in any preceding claim characterised in that the patch (3) is of an opaque material.

18. An eye shield as claimed in any preceding claim characterised in that the patch (3) defines an outer periphery (4) which is of substantially similar shape to the oval outline shape of an eye cavity.

19. An eye shield as claimed in claim 18 characterised in that the oval shaped patch (3) defines a major axis (5), which in use extends substantially parallel to a line defined by the abutting edges of the upper and lower eyelids of the eye when closed.

20. An eye shield as claimed in claim 18 or 19 characterised in that the patch (3) defines an upper straight edge (21) for preventing contact of the patch with the eyebrow of the eye.

21. An eye shield as claimed in claim 20 characterised in that the upper straight edge (21) of the patch (3) extends parallel to the major axis (5) of the patch (3).

22. An eye shield as claimed in any of claims 19 to 21 characterised in that the viewing means (24) defines an upper straight edge (30) which extends parallel to the major axis (5) of the patch (3).

23. An eye shield as claimed in claim 22 characterised in that the upper straight edge (30) of the viewing means (24) lies above the major axis (5) of the patch (3).

24. An eye shield as claimed in claim 23 characterised in that the viewing means (24) is of semi-oval shape, extending downwardly from the upper edge (30) thereof.

25. An eye shield as claimed in any of claims 18 to 24 characterised in that the patch is of substantially similar size to the size of the eye cavity.

26. An eye shield as claimed in any preceding claim characterised in that a protective release sheet (34,35) is provided for covering and protecting the adhesive means (11,12).

27. An eye shield as claimed in claim 26 characterised in that the protective release sheet (34,35) is provided in two parts which meet adjacent the major axis (5) of the patch (3), and adjacent edges of the release sheets extend parallel to the major axis of the patch.

28. An eye shield as claimed in claim 27 characterised in that the respective release sheets (34,35) overlap adjacent the major axis (5) of the patch (3).

29. An eye shield as claimed in any preceding claim characterised in that the adhesive means (11,12) is a self adhesive provided on the respective upper and lower adhesive portions (14,15).

30. An eye shield as claimed in any preceding claim characterised in that the adhesive means (11,12) is a non-irritant adhesive.

31. An eye shield as claimed in any preceding claim characterised in that the adhesive means (11,12) is a medical grade adhesive.

32. An eye shield as claimed in any preceding claim characterised in that the patch (3) is of a flexible material.

33. An eye shield as claimed in any preceding claim characterised in that the patch (3) is of a relatively thin film material.

34. An eye shield as claimed in any preceding claim characterised in that the patch and the window are of a material substantially impermeable to chemicals and other solutions used in a surgical procedure or in the preparation of a subject for a surgical procedure.

35. A pair of eye shields as claimed in any preceding claim.

36. An eye shield for protecting an eye of a subject, characterised in that the eye shield (1) comprises a patch (3) defining an outer periphery (4) which is of substantially similar shape to the oval outline of the shape of an eye cavity, and having a rear major surface (7) for in use abutting at least an upper lid of an eye of the subject, and a front major surface (6), an adhesive means (11,12) on the rear major surface (7) for securing the patch (3) to the upper eyelid and to a lower eyelid or the portion of the face of the subject below the eye for retaining the eyelids closed.

37. An eye shield as claimed in claim 36 characterised in that the apportion of the rear major surface of the patch (3) defines an intermediate area (18) for in use coinciding with eyelashes of the subject, the intermediate area (18) being such as to facilitate removal of the eye shield without plucking of the eyelashes, and with minimum discomfort to the subject.

38. An eye shield as claimed in claim 37 characterised in that the intermediate area (18) is provided by a non-adhesive surface (18) for preventing adhesion of the patch to the eye lashes of the respective upper and lower eyelids.

39. An eye shield as claimed in claim 37 or 38 characterised in that the adhesive means (11) is provided on an upper portion of the rear major surface which defines an upper adhesive portion (14) for securing the patch to the upper eyelid intermediate the eyebrow and eyelash, and the adhesive means (12) is provided on a lower portion of the rear major surface which defines a lower adhesive portion (15) for securing the patch to the lower eyelid or to the face of the subject below the eye, the intermediate area (18) being located between the upper and lower adhesive portions (14,15).

40. An eye shield as claimed in claim 39 characterised in that the adhesive means (12) is located on the lower adhesive portion (15,25) of the rear major surface (7) for securing the patch (3) below the eyelash of the lower eyelid.

41. An eye shield as claimed in any of claims 39 or 40 characterised in that the intermediate area (18) extends from one side edge (19) to another opposite side edge (20) of the patch between the upper and lower adhesive portions (14,15) thereof.

42. An eye shield as claimed in any of claims 39 to 41 characterised in that the lower adhesive portion (15) of the patch comprises an adhesive tab (16) extending downwardly therefrom for securing to the face of the subject below the eye.

43. An eye shield as claimed in claim 42 characterised in that the adhesive tab (16) extends obliquely downwardly from the patch.

44. An eye shield as claimed in claim 42 or 43 characterised in that a pair of adhesive tabs (16) diverging outwardly from each other extend downwardly from the patch.

45. An eye shield as claimed in any of claims 36 to 44 characterised in that the upper adhesive portion (14) defines an area which is substantially similar to the shape of an upper eyelid when closed.

46. An eye shield as claimed in any of claims 36 to 45 characterised in that a non-adhesive tab (32) extends from the patch (3), and preferably upwardly from the patch (3) for facilitating removal of the patch from the eye, preferably, in a direction from the upper eyelid downwardly towards the lower eyelid for minimising discomfort to the subject during removal thereof.

47. An eye shield as claimed in any of claims 36 to 46 characterised in that the patch (3) defines a major axis (5), which in use extends substantially parallel to a line defined by the abutting edges of the upper and lower eyelids of the eye when closed.

48. An eye shield as claimed in any of claims 36 to 47 characterised in that the patch (3) defines an upper straight edge (21) for preventing contact of the patch (3) with the eyebrow of the eye.

49. An eye shield as claimed in any of claims 36 to 48 characterised in that a protective release sheet (34,35) is provided for covering and protecting the adhesive means (11,12).

50. An eye shield as claimed in claim 49 characterised in that the protective release sheet (34,35) is provided in two parts which meet adjacent the major axis (5) of the patch (3), and adjacent edges of the release sheets extend parallel to the major axis of the patch.

51. An eye shield as claimed in claim 50 characterised in that the respective release sheets (34,35) overlap adjacent the major axis (5) of the patch.

52. An eye shield as claimed in any of claims 36 to 51 characterised in that the patch is of substantially similar size to the size of the eye cavity.

53. An eye shield as claimed in any of claims 36 to 52 characterised in that the adhesive means (11,12) is a self adhesive provided on the respective upper and lower adhesive portions (14,15).

54. An eye shield as claimed in any of claims 36 to 53 characterised in that the adhesive means (11,12) is a non-irritant adhesive.

55. An eye shield as claimed in any of claims 36 to 54 characterised in that the adhesive means (11,12) is a medical grade adhesive.

56. An eye shield as claimed in any of claims 36 to 55 characterised in that the patch (3) is of a flexible material.

57. An eye shield as claimed in any of claims 36 to 56 characterised in that the patch (3) is of a relatively thin film material.

58. An eye shield as claimed in any of claims 36 to 57 characterised in that the patch and the window are of a material substantially impermeable to chemicals and other solutions used in a surgical procedure or in the preparation of a subject for a surgical procedure.

59. A pair of eye shields as claimed in any of claims 36 to 58.

60. A method for protecting an eye of a subject during a surgical procedure comprising the steps of securing a patch (3) to an upper lid of the eye of the subject, urging the upper lid downwardly for closing the eye and securing the patch (3) to the lower lid of the eye, or to a portion of the face of the subject below the eye for retaining the upper and lower lids of the eye closed.

61. A method as claimed in claim 60 characterised in that the method further comprises the step of inspecting the eyelids through a viewing means (24) in the patch (3) for determining the open/closed status of the eyelids.

62. A method as claimed in claim 60 or 61 characterised in that the method further comprises preventing securing of the patch (3) to eyelashes of the respective upper and lower eyelids when securing the patch to the upper eyelid and to the lower eyelid or the portion of the face below the eye.

63. A method as claimed in any of claims 60 to 62 characterised in that the method comprises securing the patch to the upper eyelid intermediate the eyelash of the upper eyelid and the eyebrow of the eye of the subject.

64. A method as claimed in any of claims 60 to 63 characterised in that the method further comprises the step of removing the patch (3) from the eye by peeling the patch (3) from the upper eyelid in a direction from the upper eyelid downwardly towards the lower eyelid.

65. A method as claimed in any of claims 60 to 64 characterised in that the method comprises the step of securing a pair of patches (3) to the respective eyes of the subject.

66. A method as claimed in any of claims 60 to 65 characterised in that the patch (3) is provided by an eye shield (1) as claimed in any of claims 1 to 59.
